(19) United States
(12) Patent Application Publication
(10) Pub. No.: US 2012/0136431 A1
(43) Pub. Date: May 31, 2012

(54) Stent Graft System

(76) Inventor: Wei-Hui Chen, Taichung (TW)

(21) Appl. No.: 12/955,291

(22) Filed: Nov. 29, 2010

(51) Int. Cl. A61F 2/82

(52) U.S. Cl. 623/1.35

(57) ABSTRACT

The stent graft system in accordance with the present invention has a trunk, a left subclavian tube, a left common carotid tube and a brachiocephalic tube. The trunk is tubular and expandable and has a descending end, an ascending end, a left subclavian mount, a left common carotid mount and a brachiocephalic mount, for receiving the aforementioned branch tubes that are elastic and self-expandable for respectively connecting the left subclavian artery, the left common carotid artery and the brachiocephalic artery to the trunk. With the above-described structure, the present invention allows fast determination of a suitable trunk and branch tubes for a patient and allows a medical institute to prepare compatible branch tubes and trunks for a composite stent graft system instead of numerous stent grafts of various combinations of differently sized tubular bodies and branches, wherein the former requires significantly less warehousing cost than the latter.

### BACKGROUND OF THE INVENTION

1. Field of the Invention

The present invention relates to a stent graft system, especially to a composite branched stent graft system.

2. Description of the Prior Art

With reference to FIG. 18, a first conventional stent graft or prosthesis for open aortic arch surgery is used to support inner wall thereof as a treatment for aortic dissection or aortic arch aneurysm and comprises a tubular trunk 91, a left subclavian arterial branch 911, a left common carotid arterial branch 912 and a brachiocephalic arterial branch 913. The tubular trunk 91 is made of self-expandable elastic material with expandable stents and comprises two ends 914, 915 for connecting to portions of the aortic arch. The two ends 914, 915 of the tubular trunk 91, when mounted in the aortic arch, self-expand and thus attach themselves to the inner walls of the portions of aortic arch. The portion of the vicinity of the end 915 also comprises the expandable stents so as to mount the trunk 91 in the ascending aorta by the expanding stents. However, the tightening made possible by the expanding stents is observed to be insufficient to endure the pressure of the blood steam from ascending aorta and fails to sustain the trunk 91. Furthermore, the expandable stents are embedded in or attached to the portions of the vicinities of the ends 914, 915 for mounting the trunk 91 in the aorta, while the rest portion of the trunk 91 are free from stents. Not being wholly embedded with reinforcing stents, the trunk 91 of the first conventional stent graft or prosthesis fails to keep the shapes of its original curves and can be forced straight when passing the blood streams.

The left subclavian arterial branch 911, the left common carotid arterial branch 912 and the brachiocephalic arterial branch 913, made of the same self-expandable elastic material with expandable stents, are irremovably originated from and communicated with the tubular trunk 91, and are respectively connected to and communicate with the left subclavian artery, the left common carotid artery and the brachiocephalic artery (innominate artery) by means of self-expansion therein.

With reference to FIG. 19, a second conventional stent graft is similar to the foregoing stent graft, except that the self-expandable elastic tubular trunk 92 is made of longitudinally interleaved mesh and spring materials. The left subclavian arterial branch 921, the left common carotid arterial branch 922 and the brachiocephalic arterial branch 923 of the second conventional stent graft are irremovably originated from a spring-material portion of the tubular trunk 92. The second conventional stent graft attaches itself to the inner walls of the aortic arch and the branches by means of self-expansion.

With further reference to FIGS. 18 and 19, the structure of each of the aforementioned conventional stent grafts provides a tubular trunk 91, 92 and irremovable branches 911-913, 921-923. There are chances that even though the tubular trunk 91, 92 is suitable for the aortic arch of the patient, all or one of the branches 911-913, 921-923 may be ill fitting for the left subclavian artery, the left common carotid artery and/or the brachiocephalic artery.

However, the diameters of aortae treated in a medical institution vary, so do the diameters of the left subclavian artery, the left common carotid artery and the brachiocephalic artery. A conventional stent graft whose trunk and all branches respectively compatible to the aorta and arteries being treated is indispensable in a surgery for aortic dissection or aortic arch aneurysm. This structural nature of the conventional stent grafts costs medical institutions a burden of preparing readily available stent grafts that come with various combinations of different sized tubular trunks 91, 92 and irremovable branches 911-913, 921-923, so to satisfy the surgical need for engaging aortae and arteries of various diameters.

One possible solution to the above-described shortcoming due to warehousing a large stock of various sized stent grafts is applying custom-made stent grafts. One apparent problem of the solution is its unacceptable expensiveness. In a clinical aspect of the custom-made stent grafts a more significant problem arises: custom-made stent grafts usually fail to provide clinically realistic readiness, especially in an emergent surgery.

Said structural nature of conventional stent grafts also costs the surgeon considerable time for selecting a stent graft with specifically suitable tubular trunks 91, 92 and suitable branches 911-913, 921-923, prior to or during an emergent surgery, such as acute aortic dissection or ruptured aortic aneurysm, in which the customized branched stent graft cannot be available timely.

To overcome the shortcomings, the present invention provides a stent graft system to mitigate or obviate the aforementioned problems.

### SUMMARY OF THE INVENTION

The main objective of the invention is to provide a stent graft system.

The stent graft system in accordance with the present invention has a trunk, a left subclavian tube, a left common carotid tube and a brachiocephalic tube.

The trunk is tubular and expandable, preferably balloon-expandable or self-expandable, and has a descending end, an ascending end, a left subclavian mount, a left common carotid mount and a brachiocephalic mount. The mounts are for receiving the aforementioned branch tubes, i.e., the left subclavian tube, the left common carotid tube and the brachiocephalic tube. The branch tubes are made of various blood-impermeable materials, especially polyesteror polytetrafluoroethylene, where expandable, preferably self-expandable or balloon-expandable, stents are mounted on their inner or outer surfaces. The stents are also embedded in a portion of the branch tube for mounting in the trunk. Said expandable stents may be made of Nitinol, stainless steel, Co—Cr alloy, or other clinically acceptable material enabling the functionality of the stents. The branch tubes are used for respectively connecting the left subclavian artery, the left common carotid artery and the brachiocephalic artery to the trunk.

With the above-described structure, the present invention allows a surgeon to separately select a suitable trunk, a left subclavian tube, a left common carotid tube and a brachiocephalic tube in the aortic arch surgery, which takes far less time than selecting a complete stent graft that coincidentally fits the aortic arch as well as the arteries of the patient. Furthermore, the structure of the stent graft system allows a medical institute to prepare compatible branch tubes and trunks for a composite stent graft system instead of numerous stent grafts of various combinations of differently sized tubular bodies and branches, wherein the former requires significantly less warehousing cost than the latter.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of the trunk of the stent graft system in accordance with the present invention;

FIG. 2 is a perspective view of a second embodiment of the trunk of the stent graft system in accordance with the present invention;

FIG. 3 is a perspective view of a modified second embodiment of the trunk of the stent graft system in accordance with the present invention;

FIG. 4 is a perspective view of a third embodiment of the trunk of the stent graft system in accordance with the present invention;

FIG. 5 is a side view of a first embodiment of a branch tube for use as a left subclavian tube, a left common carotid tube or a brachiocephalic tube of the stent graft system in accordance with the present invention;

FIG. 6 is a side view of a second embodiment of a branch tube for use as a left subclavian tube, a left common carotid tube or a brachiocephalic tube of the stent graft system in accordance with the present invention;

FIG. 7 is a side view of a third embodiment of a branch tube for use as a left subclavian tube, a left common carotid tube or a brachiocephalic tube of the stent graft system in accordance with the present invention;

FIG. 8 is a side view of a forth embodiment of a branch tube for use as a left subclavian tube, a left common carotid tube or a brachiocephalic tube of the stent graft system in accordance with the present invention;

FIG. 9 is a side view of a fifth embodiment of a branch tube for use as a left subclavian tube, a left common carotid tube or a brachiocephalic tube of the stent graft system in accordance with the present invention;

FIG. 10 is a side view of a sixth embodiment of a branch tube for use as a left subclavian tube, a left common carotid tube or a brachiocephalic tube of the stent graft system in accordance with the present invention;

FIG. 11 is a side view of a seventh embodiment of a branch tube for use as a left subclavian tube, a left common carotid tube or a brachiocephalic tube of the stent graft system in accordance with the present invention;

FIG. 12 is a side view of an eighth embodiment of a branch tube for use as a left subclavian tube, a left common carotid tube or a brachiocephalic tube of the stent graft system in accordance with the present invention;

FIG. 13 is a side view of a ninth embodiment of a branch tube for use as a left subclavian tube, a left common carotid tube or a brachiocephalic tube of the stent graft system in accordance with the present invention;

FIG. 14 is an exploded operational perspective view of a second example of the stent graft system in accordance with the present invention;

FIG. 15 is an exploded operational perspective view of a first example of the stent graft system in accordance with the present invention;

FIG. 16 is an operational perspective view of a trunk in FIG. 1 connected to the aorta with a fastening belt;

FIG. 17 is an operational perspective view of a trunk in FIG. 1 connected to the aorta with another fastening belt;

FIG. 18 is a perspective view of a conventional stent graft in accordance with the prior art; and

FIG. 19 is a side view of another conventional stent graft in accordance with the prior art.

### DETAILED DESCRIPTION OF THE PREFERRED EMBODIMENTS

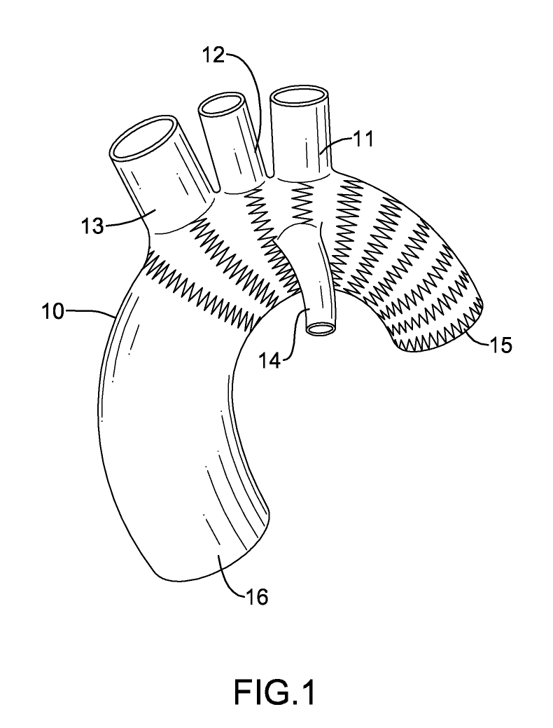

With reference to FIGS. 1, 5, 7, 8 and 15, a first example of the stent graft system in accordance with the present invention has a trunk 10, a left subclavian tube 41, a left common carotid tube 43 and a brachiocephalic tube 46.

The trunk 10 is tubular and expandable, preferably self-expandable or balloon-expandable. The trunk 10 comprises expandable stents embedded therein or mounted thereon, preferably made of meshes or springs or a combination thereof, wherein the material of the stents may be selected from various clinically acceptable materials, especially Nithnol, stainless steel or Co—Cr alloy. The trunk 10 comprises a descending end 15, an ascending end 16, a left subclavian mount 11, a left common carotid mount 12 and a brachiocephalic mount 13. The trunk 10 is used with its self-expanding or balloon-expandable feature to support the inner wall of the aorta as a treatment for aortic dissection or aortic arch aneurysm. The ascending and descending ends 16, 15 are respectively orientated to the ascending aorta and descending aorta within the aortic arch so to receive the blood flow from the ascending end 16 and distribute the same to the descending aorta through the descending end 15, to the left subclavian artery (S) through the left subclavian mount 11, to the left common carotid artery (L) through the left common carotid mount 12 and to the brachiocephalic artery (B) through the brachiocephalic mount 13.

The left subclavian mount 11, the left common carotid mount 12 and the brachiocephalic mount 13 have respectively predefined diameter and are solid for receiving the left subclavian tube 41, the left common carotid tube 43 and the brachiocephalic tube 46.

In the present example, the trunk 10 further comprises a control duct 14 originated from and communicated with the trunk 10, allowing external access to the trunk 10, the left subclavian tube 41, the left common carotid tube 43 and the brachiocephalic tube 46 and switching the same into expanded status. The self-expandable branch tubes 41, 43, 46 respectively inserted in the mounts 11-13 firmly attach themselves therein when switched into expanded status. The control duct 14 is also used as an inlet for inducing blood to initiate a cardio-pulmonary bypass.

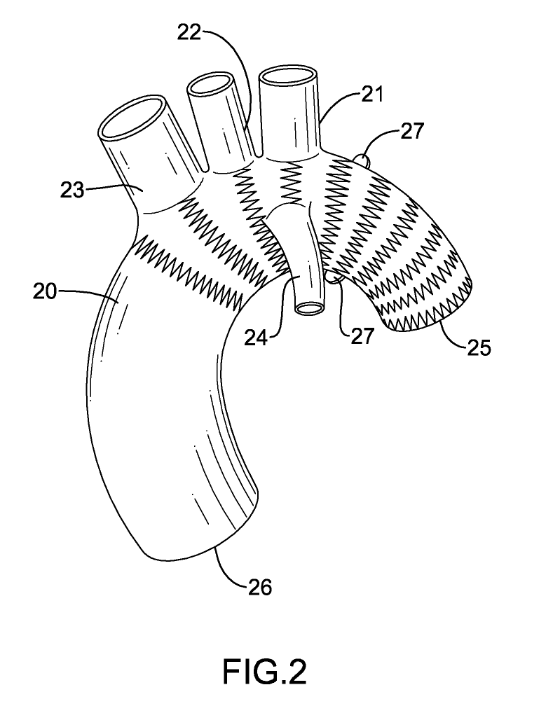

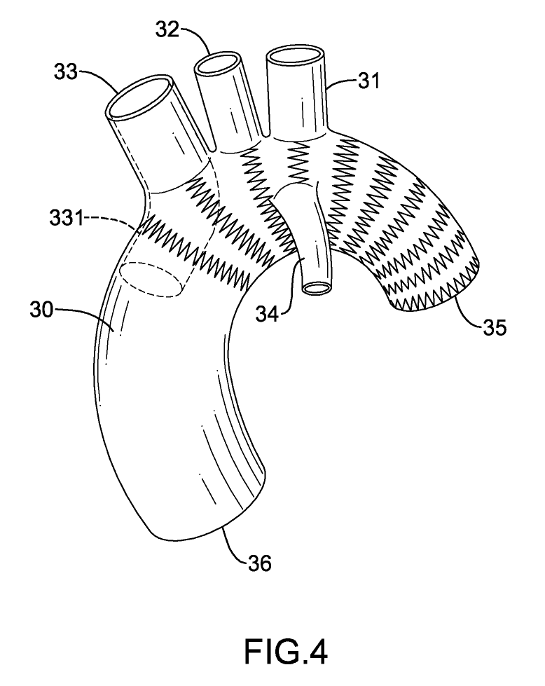

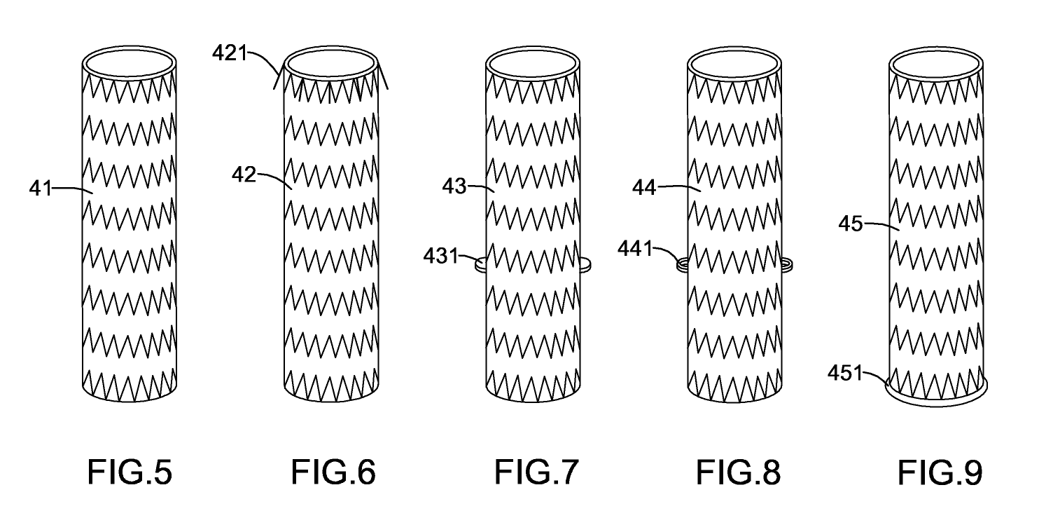

With further reference to FIGS. 5-12, branch tubes 41, 42, 43, 46, 47, 48 of various structures are available options for forming a functional stent graft with the trunk 10 or with other embodiments of the trunk 20, 30 as shown in FIGS. 2 and 4. The aforementioned first example, though using specific branch tubes 41, 42, 43 with the trunk 10, is for describing one of various feasible functioning embodiments of the stent graft system in accordance with the present invention, instead of for limiting the scope thereof in any aspect.

With reference to FIG. 2, the second embodiment of the trunk 20 is structurally and functionally similar to the first embodiment and also comprises a descending end 25, an ascending end 26, a left subclavian mount 21, a left common carotid mount 22, a brachiocephalic mount 23 and a control duct 24. The trunk 20 comprises an outer surface and multiple tabs 27. The tabs 27 are attached to the outer surface between the left subclavian mount and the descending end of the trunk 20 for suturing the trunk 20 to the aortic arch, so to provide firm combination of the trunk 20 to the aortic arch. The trunk 20 may comprise multiple rings 28 or a combination of rings and tabs 27. The rings 28, through which seaming materials and apparatus are allowed to pass, are attached to the outer surface of the trunk 20 and function similarly as the tabs 27. Both the tabs 27 and the rings 28 help to prevent slippage of the trunk 20 from the aortic arch.

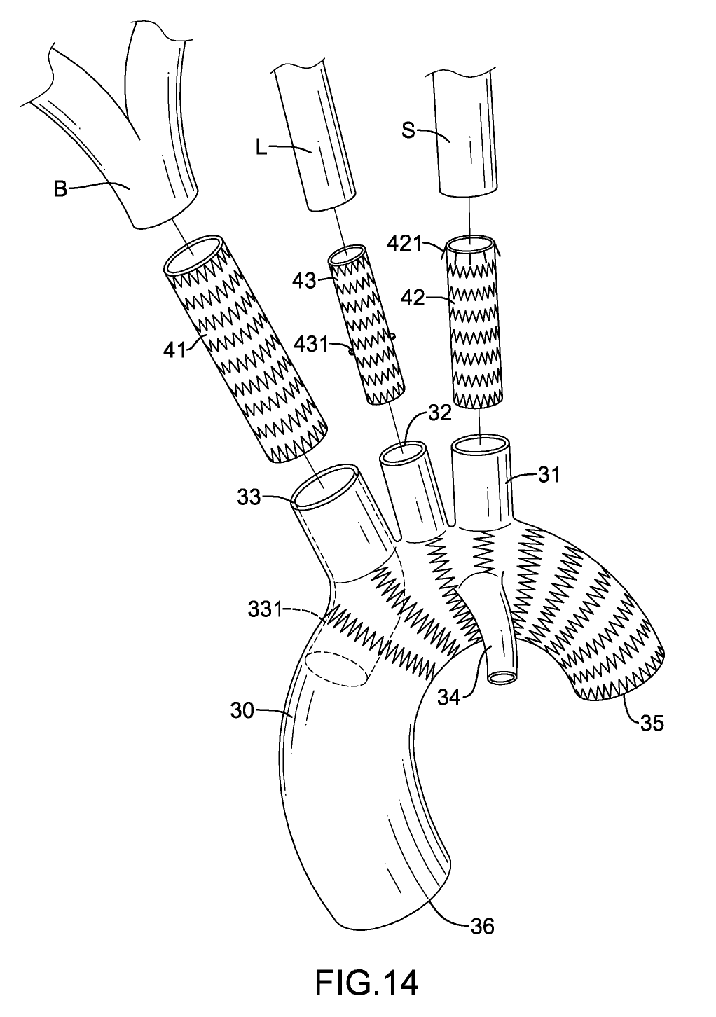

With reference to FIGS. 4 and 14, the third embodiment of the trunk 30 is structurally and functionally similar to the first embodiment and also comprises a descending end 35, an ascending end 36, a left subclavian mount 31, a left common carotid mount 32, a brachiocephalic mount 33 and a control duct 34. The brachiocephalic mount 33 extends within the trunk 30 and comprises an inner end 331. The inner end 331 points to the ascending end 36 of the trunk 30 in order to reinforce the junction portion between the trunk 30 and the brachiocephalic tube 41 so to better endure the pressure brought by the strong blood flow received therein through the ascending end 36.

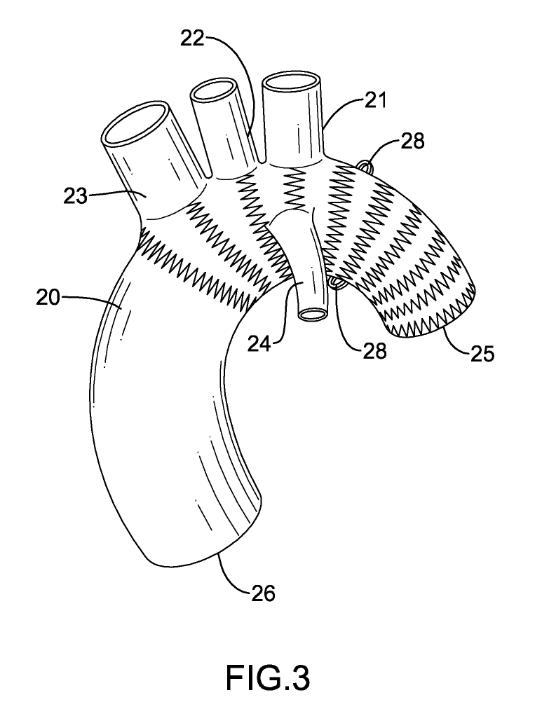

With reference to FIG. 5, a first embodiment of a branch tube 41 for use as a left subclavian tube, a left common carotid tube or a brachiocephalic tube of the stent graft system in accordance with the present invention is expandable, preferably self-expandable or balloon-expandable made from a clinically blood-impermeable material, preferably polyester or polytetrafluoroethylene, where expandable stents are mounted on inner or outer surfaces of the branch tube 41. The expandable stents may be made of meshes, springs or a combination thereof, and comprises a proximal end and a distal end. Preferably, the material of the stents may be Nitinol, stainless steel, Co—Cr alloy, or other clinically acceptable material. With reference to FIGS. 1-4, 14 and 15, the proximal end of the branch tube 41 is for mounting the left subclavian mount 11, 21, 31, the left common carotid mount 12, 22, 32 or the brachiocephalic mount 13, 23, 33. The distal end is for connecting to the left subclavian artery, the left common carotid artery or the brachiocephalic artery.

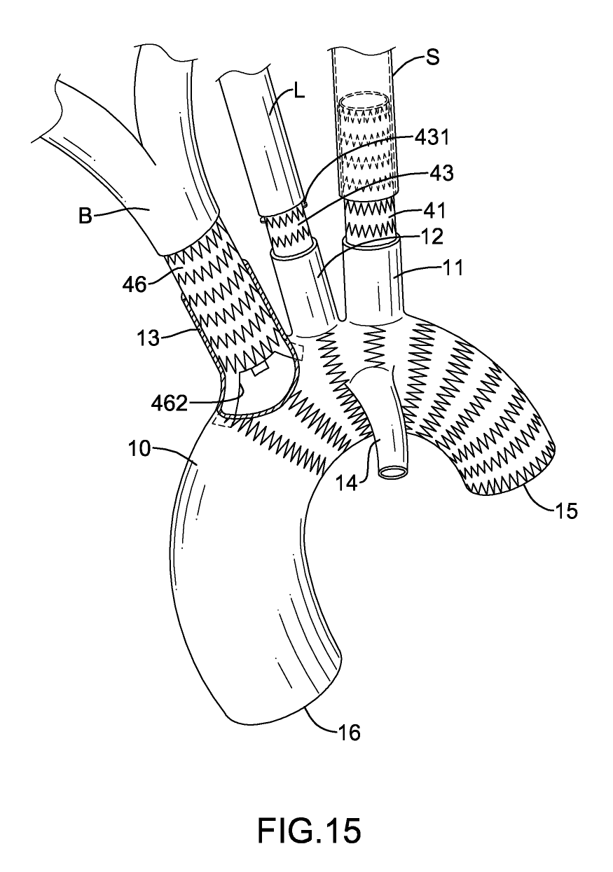

With reference to FIG. 15, when used as a left subclavian tube for instance, after inserted in the left subclavian mount 11 of the trunk 10 and in the left subclavian artery, the branch tube 41 is maneuvered to expand, especially by means of external access through the control duct 14, so to function as a subclavian tube. The expanded tube 41 then firmly attaches itself to the inner walls of the left subclavian artery and the left subclavian mount 11, in order to pass blood flow from the trunk 10 to the left subclavian artery.

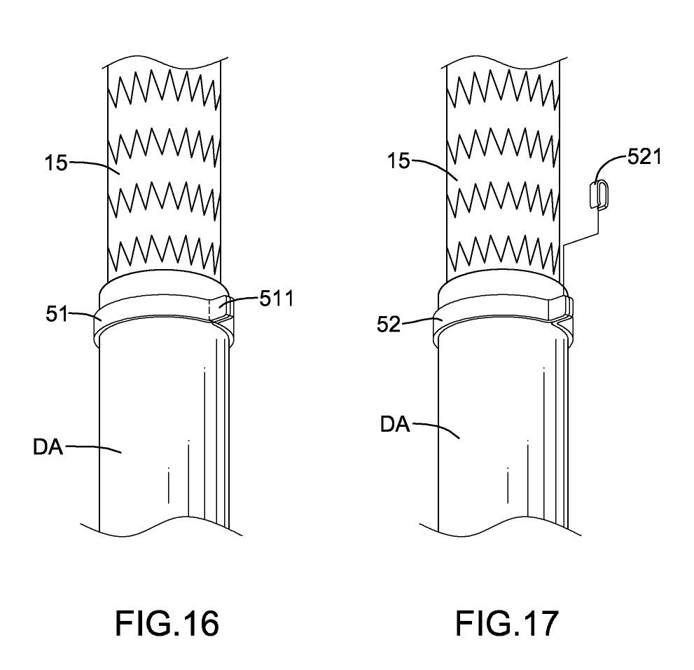

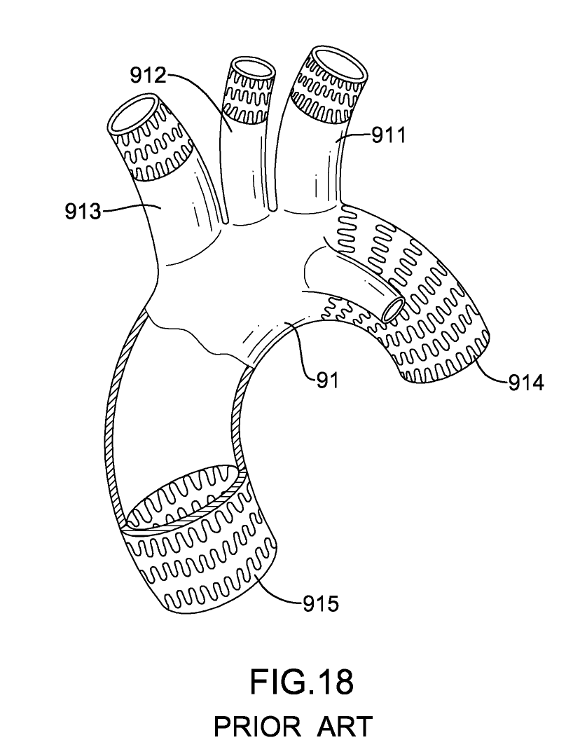

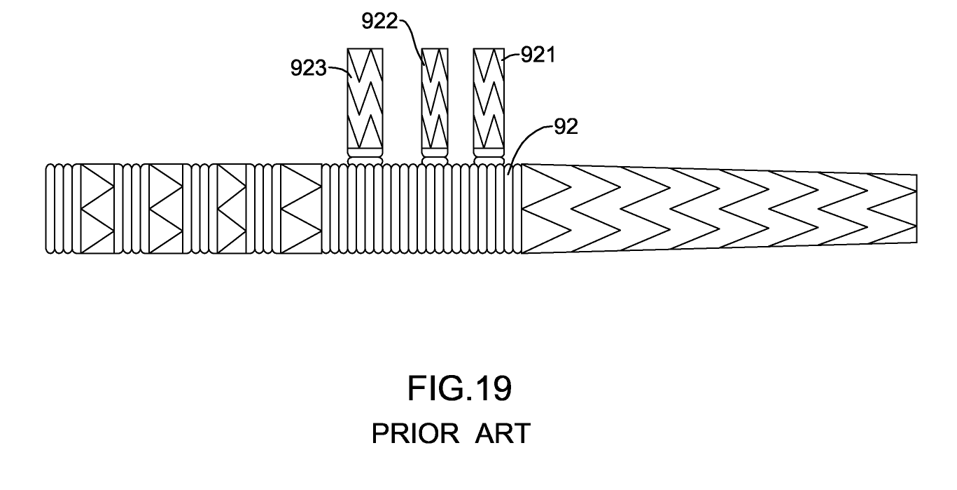

With reference to FIG. 16, an additional fastener may be employed to further secure the connection of the descending aorta (DA) to the trunk 10 at the descending end 15. One example of said additional fastener is a fastening belt 51 with attached ends 511. With reference to FIG. 17, another example of said additional fastener is a fastening belt 52 whose ends are pinched with a pincher 521.

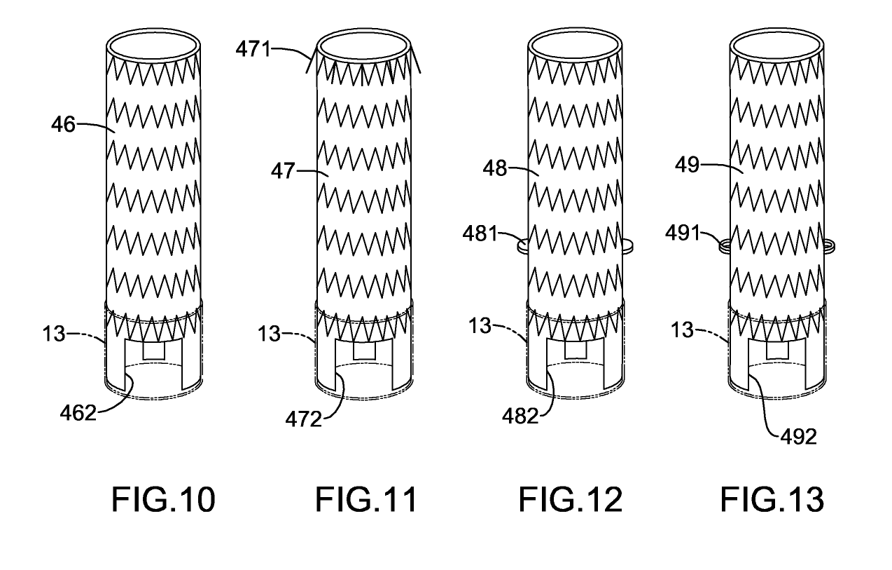

With reference to FIG. 6, a second embodiment of a branch tube 42, which is structurally and functionally similar to the first embodiment, comprises an outer surface and multiple barbs 421. The barbs 421 are circumferentially attached to the outer surface around the distal end of the branch tube 42. When attached to the inner wall of an artery, the barbs 421 further secure the connection of the branch tube 42 and the artery, wherein the artery may be the left subclavian artery, the left common carotid artery or the brachiocephalic artery. With reference to FIG. 10, when the branch tube 42 is used as a left subclavian tube, the barbs 421 hook the inner surface of the subclavian artery to prevent detachment thereof.

With reference to FIG. 7, a third embodiment of a branch tube 43, which is structurally and functionally similar to the first embodiment, comprises an outer surface and multiple tabs 431. The tabs 431 are attached to the outer surface between the distal end and the proximal end of the left subclavian tube. When the branch tube 43 is inserted in an artery, the surgeon may suture the tabs with the artery to provide secured connection between the branch tube 43 and the artery. With reference to FIGS. 14 and 15, when the branch tube 43 is used as a left common carotid tube, attachment of the tabs 431 to the left common carotid artery by use of sutures is preferred. With further reference to FIG. 4, the branch tube 43 is also suitable for mounting in the left subclavian mount 31, the left common carotid mount 32 or the brachiocephalic mount 33 of the trunk 30.

With reference to FIG. 8, a forth embodiment of a branch tube 44 similar to the aforementioned third embodiment comprises an outer surface and multiple rings 441. The rings 441, attached to the outer surface of the branch tube 44 in a similar manner as the tabs 431 to the branch tube 43 in FIG. 7, allow seaming materials to pass through them, so to provide convenience in surgery.

With further reference to FIGS. 1-4 and 6, a similar embodiment to the foregoing second embodiment of the branch tube 42 further comprises a reinforce ring formed on the outer surface around the proximal end of the branch tube 42. The reinforced ring is elastic and capable of being deformed and compressed when mounted in a mount 11-13, 21-23, 31-33 of a trunk 10, 20, 30. When the instant embodiment is deployed in the mount 11-13, 21-23, 31-33 and one of the three supra-aortic arteries, the ring is fully expanded and can be engaged on the margin of mount.

With further reference to FIGS. 1-4, 5 and 9, the present invention also provides a fifth embodiment of a branch tube 45 similar to the aforementioned embodiment of the branch tube 41, wherein no barbs as mentioned in the second embodiment of the branch tube 42, however equipped with the above-described reinforced ring 451 formed on the outer surface around the proximal end.

With reference to FIG. 10, a sixth embodiment of a branch tube 46, which is structurally and functionally similar to the first embodiment, comprises a tubular wall and at least one notch 462. Each notch 462 is indented from the proximal end allowing the proximal end of the branch tube 46 to form an efficiently receiving opening. With reference to FIG. 15, the branch tube 46, suitable for using as a left subclavian arterial tube or a left common carotid arterial tube mounted in a corresponding mount, is also capable of being deployed as a brachiocephalic tube that mounted in the brachiocephalic mount 13, so that the at least one notches 462 forms a stable and secure proximal fixation of the branch tube 46 in the trunk 10.

With reference to FIGS. 11 and 12, the seventh and eighth embodiments of branch tubes 47, 48, both having at least one notch 472, 482, and respectively multiple barbs 471 and multiple tabs 481, are structurally and functionally similar to the sixth embodiment. The multiple barbs 471 have similar structure and arrangement as the barbs 421 of the second embodiment. The multiple tabs 481 have similar structure and arrangement as the tabs 431 of the third embodiment.

With reference to FIG. 13, a ninth embodiment of a branch tube 49 similar to the aforementioned eighth embodiment comprises at least one notch 492, an outer surface and multiple rings 491. The rings 491, attached to the outer surface of the branch tube 49 in a similar manner as the tabs 481 to the branch tube 48 in FIG. 12, allow seaming materials to pass through them, so to provide convenience in surgery.

With further reference to FIGS. 1-13, each of the embodiments of the branch tubes described above, including branch tubes 41-49, are capable of being mounted in any of the mounts 11-13, 21-23, 31-33 formed on a trunk 10, 20, 30 of the stent graft system in accordance with the present invention. A surgeon may select a suitable trunk 10, 20, 30 for a patient and determine suitable branch tubes according to the diameters and conditions of the arteries thereof in a reasonable short time. The stent graft system of the above-described structure provides a time saving means for surgeons to rapid select suitable branch tubes and trunks and is also economic and efficient for warehousing management of medical institutes.

What is claimed is:

1. A stent graft system comprising
a trunk being tubular and expandable and comprising
reinforcing stents;
a descending end for connecting a portion of the aortic arch orientated to the descending aorta;
an ascending end for connecting a portion of the aortic arch orientated to the ascending aorta;
a solid left subclavian mount;
a solid left common carotid mount; and
a solid brachiocephalic mount;
a left subclavian tube for connecting the left subclavian artery to the trunk, being expandable and mounted in the left subclavian mount of the trunk and comprising
a proximal end for mounting the left subclavian mount of the trunk; and
a distal end for connecting to the left subclavian artery;
a left common carotid tube for connecting the left common carotid artery to the trunk, being expandable and mounted in the left common carotid mount of the trunk and comprising
a proximal end for mounting the left common carotid mount of the trunk; and
a distal end for connecting to the left common carotid artery; and
a brachiocephalic tube for connecting the brachiocephalic artery to the trunk, being expandable and mounted in the brachiocephalic mount of the trunk and comprising
a proximal end for mounting the brachiocephalic mount of the trunk; and
a distal end for connecting to the brachiocephalic artery.

2. The stent graft system as claimed in claim 1, wherein
the trunk further comprises a control duct originated from and communicated with the trunk, allowing external access to the trunk, the left subclavian tube, the left common carotid tube and the brachiocephalic tube.

3. The stent graft system as claimed in claim 1, wherein
the trunk further comprises a control duct originated from and communicated with the trunk, allowing external access and cardiopulmonary bypass inflow to the trunk, the left subclavian tube, the left common carotid tube and the brachiocephalic tube.

4. The stent graft system as claimed in claim 1, wherein
the trunk comprises
an outer surface; and
multiple tabs attached to the outer surface between the left subclavian mount and the descending end of the trunk and for suturing the trunk to the aortic arch.

5. The stent graft system as claimed in claim 1, wherein
the trunk comprises
an outer surface; and
multiple rings attached to the outer surface between the left subclavian mount and the descending end of the trunk and for suturing the trunk to the aortic arch.

6. The stent graft system as claimed in claim 1, wherein
the brachiocephalic mount extends within the trunk and comprises
an inner end pointing to the ascending end of the trunk.

7. The stent graft system as claimed in claim 1, wherein
the left subclavian tube is selected from the group consisting of
a) a branch tube further comprising
an outer surface; and
multiple barbs circumferentially attached to the outer surface around the distal end of the left subclavian tube;
b) a branch tube further comprising
an outer surface; and
multiple tabs attached to the outer surface between the distal end and the proximal end of the left subclavian tube; and
c) a branch tube further comprising
an outer surface; and
multiple rings attached to the outer surface between the distal end and the proximal end of the left subclavian tube.

8. The stent graft system as claimed in claim 6, wherein
the left subclavian tube comprises
a tubular wall; and
at least one notch indented from the proximal end allowing the proximal end of the left subclavian tube to form a stable and secure proximal fixation of the left subclavian tube in the trunk.

9. The stent graft system as claimed in claim 1, wherein
the left subclavian tube is selected from the group consisting of
a) a branch tube further comprising
an outer surface;
multiple barbs circumferentially attached to the outer surface around the distal end of the left subclavian tube; and
a reinforce ring formed on the outer surface around the proximal end of the left subclavian tube; and
b) a branch tube further comprising
an outer surface; and
a reinforce ring formed on the outer surface around the proximal end of the left subclavian tube;

10. The stent graft system as claimed in claim 1, wherein
the left common carotid tube is selected from the group consisting of
a) a branch tube further comprising
an outer surface; and
multiple barbs circumferentially attached to the outer surface around the distal end of the left common carotid tube;
b) a branch tube further comprising
an outer surface; and
multiple tabs attached to the outer surface between the distal end and the proximal end of the left common carotid tube; and
c) a branch tube further comprising
an outer surface; and
multiple rings attached to the outer surface between the distal end and the proximal end of the left common carotid tube.

11. The stent graft system as claimed in claim 10, wherein
the left common carotid tube comprises
a tubular wall; and
at least one notch indented from the proximal end allowing the proximal end of the left common carotid tube to form a stable and secure proximal fixation of the left common carotid tube in the trunk.

12. The stent graft system as claimed in claim 1, wherein
the left common carotid tube is selected from the group consisting of
a) a branch tube further comprising
an outer surface;
multiple barbs circumferentially attached to the outer surface around the distal end of the left common carotid tube; and
a reinforce ring formed on the outer surface around the proximal end of the left common carotid tube; and
b) a branch tube further comprising
an outer surface; and
a reinforce ring formed on the outer surface around the proximal end of the left common carotid tube;

13. The stent graft system as claimed in claim 1, wherein
the brachiocephalic tube is selected from the group consisting of
a) a branch tube further comprising
an outer surface; and
multiple barbs circumferentially attached to the outer surface around the distal end of the brachiocephalic tube;
b) a branch tube further comprising
an outer surface; and
multiple tabs attached to the outer surface between the distal end and the proximal end of the brachiocephalic tube; and
c) a branch tube further comprising
an outer surface; and
multiple rings attached to the outer surface between the distal end and the proximal end of the brachiocephalic tube.

14. The stent graft system as claimed in claim 13, wherein
the brachiocephalic tube comprises
a tubular wall; and
at least one notch indented from the proximal end allowing the proximal end of the brachiocephalic tube to form a stable and secure proximal fixation of the brachiocephalic tube in the trunk.

15. The stent graft system as claimed in claim 1, wherein
the brachiocephalic tube is selected from the group consisting of
a) a branch tube further comprising
an outer surface;
multiple barbs circumferentially attached to the outer surface around the distal end of the brachiocephalic tube; and
a reinforce ring formed on the outer surface around the proximal end of the brachiocephalic tube; and
b) a branch tube further comprising
an outer surface; and
a reinforce ring formed on the outer surface around the proximal end of the brachiocephalic tube;

16. The stent graft system as claimed in claim 1, wherein
the left common carotid tube comprises
an outer surface; and
multiple tabs attached to the outer surface between the distal end and the proximal end of the left common carotid tube; and
the brachiocephalic tube comprises
a tubular wall; and
at least one notch indented from the proximal end allowing the proximal end of the brachiocephalic tube to form a stable and secure proximal fixation of the brachiocephalic tube in the trunk.

17. The stent graft system as claimed in claim 6, wherein
the left subclavian tube comprises
an outer surface; and
multiple barbs circumferentially attached to the outer surface around the distal end of the left subclavian tube; and
the left common carotid tube comprises
an outer surface; and
multiple tabs attached to the outer surface between the distal end and the proximal end of the left common carotid tube.

18. The stent graft system as claimed in claim 1, wherein the trunk is self-expandable or balloon-expandable.

19. The stent graft system as claimed in claim 1, wherein the stents are embedded in or mounted to the trunk.

20. The stent graft system as claimed in claim 1, wherein
the left subclavian tube is made from a clinically blood-impermeable material and comprises
an inner surface; and
expandable stents mounted on the inner surface or the outer surface of the left subclavian tube;
the left common carotid tube is made from a clinically blood-impermeable material and comprises
an inner surface; and
expandable stents mounted on the inner surface or the outer surface of the left common carotid tube; and
the brachiocephalic tube is made from a clinically blood-impermeable material and comprises
an inner surface; and
expandable stents mounted on the inner surface or the outer surface of the brachiocephalic tube.
